(19) United States
(12) Patent Application Publication
(10) Pub. No.: US 2009/0162443 A1
(43) Pub. Date: Jun. 25, 2009

(54) COMPOSITIONS AND METHODS FOR REDUCING OR PREVENTING WATER LOSS FROM THE SKIN

(75) Inventors: Frank A. Anthony, Memphis, TN (US); Anna Knapp Erixon, Hollister, MO (US); Courtney Denise Wright, Memphis, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc.

(21) Appl. No.: 12/338,033

(22) Filed: Dec. 18, 2008

Related U.S. Application Data
(60) Provisional application No. 61/015,895, filed on Dec. 21, 2007.

(51) Int. Cl. A61K 9/14; A61K 33/32; A61K 8/18; A61Q 17/04

(52) U.S. Cl. 424/489; 424/642; 424/59

(57) ABSTRACT

Moisturizing compositions comprising microspheres for the purpose of preventing or reducing moisture loss from the skin.

Some example embodiments of the present invention are generally directed to compositions for and methods of reducing or preventing water loss from the skin. This application claims priority from U.S. provisional patent application Ser. No. 61/015,895 filed Dec. 21, 2007.

### FIELD OF THE INVENTION

### Background

Skin barrier function is attributed to the stratum corneum, the top surface layers of the skin. The stratum corneum serves as an important interface between the environment and the human body and performs many functions including protection against microorganisms, toxic substances and loss of water. Skin surface moisture can be measured using a Corneometer that quantifies the moisture content of the stratum corneum using an electrical capacitance method. Disruptions of skin barrier function can be measured using a skin bioinstrumentation technique called trans-epidermal water loss (TEWL). When the skin is compromised or disrupted as with dry, cracked or fissured skin, there is a higher than normal water loss. When the skin barrier function is preserved or enhanced, the water loss is prevented or reduced.

The skin normally is capable of maintaining adequate internal moisture to remain pliable. Various factors, including the presence of lipid components in the skin, work to maintain proper moisture levels for most aspects of normal living. However, exposure to external moisture sources can cause a depletion of skin lipids and a consequential dermatitis. This condition is aggravated when the external moisture also contains irritating chemicals, such as are present in body wastes.

There are commercially available products for coating the skin to prevent deleterious contact with chemical substances. Such products are generally called “barrier” creams, lotions or ointments and are based on impervious substances, such as petrolatum, silicone greases, heavy oils, waxes and the like. Unfortunately, these materials leave a very greasy, sticky or oily uncomfortable coating on the skin and therefore are best suited as substitutes for vinyl or rubber gloves, applied just prior to exposure to detergents, industrial chemicals, etc.

For many uses, many persons would prefer a substance similar to those lotions, creams and the like normally used for the typical non-barrier skin care products. Such non-barrier skin care products are commonly emulsions of the oil-in-water type. Since the skin absorbs oil-in-water formulations more readily than it does all oil or water-in-oil external formulations, they tend not to cause a greasy or oily feel after application of the product. Eliminating this oily or greasy feel may be particularly desirable for application of products to the foot or to pubic and perianal regions of the body, where the skin tends to be enclosed for long periods.

### SUMMARY

Microspheres, which are commonly added to sunscreen products to enhance their sun protection (SPF) properties, have been found to have the unexpected effect of improving skin barrier function. Some example embodiments of the present invention are directed to moisturizers compositions using microspheres for the purpose of preventing or reducing moisture loss from the skin, as opposed to their more conventional use as SPF enhancers. Using the microspheres in these products is believed to enhance moisture barrier function, producing better moisturizer performance in these compositions, while allowing the elimination or reduced use of occlusive agents which may cause a greasy or oily feel. Some example embodiments include diaper creams and adult incontinence products, sunless tanning agents, antifungal creams, and foot balms or creams, and other moisturizers without sunscreen actives, and particularly without organic sunscreen actives.

One example embodiment of the invention presented herein may be a composition for preventing moisture loss from the skin which includes a moisturizing component and microspheres combined with the moisturizing component, and which does not contain UV organic actives. Another example embodiment may be a diaper cream or adult incontinence composition which contains a moisturizer containing microspheres, each potentially including zinc oxide up to 25% by weight. Another example embodiment may be a sports cream or antifungal or jock-itch cream which contains a moisturizer containing microspheres and further containing an antifungal agent. An additional example may be a sunless tanning lotion including a moisturizer containing microspheres and further comprising a skin coloring agent. Another example embodiment may be a foot balm moisturizer containing microspheres.

Another example embodiment of the invention is a composition for preventing moisture loss from the skin. The composition may include a moisturizing component; and microspheres combined with the moisturizing component, wherein the composition does not have UV organic actives. Optionally, the composition may be a diaper cream including zinc oxide up to 25% by weight. Alternatively, the composition may be a sunless tanning lotion including a skin coloring agent that optionally may be dihydroxyacetone. As a further alternative, the composition may be a foot balm including urea or other skin softener. In another alternative, the composition may be a jock-itch cream including an antifungal agent. Any of the above alternative compositions may include microspheres that are copolymers of polyalkenes and acrylates, in particular copolymers of styrene and methacrylate. In some embodiments, the microspheres may be hollow, with an average particle size of 325 nm.

Another alternative example embodiment of the present invention is a composition for preventing moisture loss from the skin including 0-3% microspheres, 0.01-1% dl-alpha tocopherol, 0.5-5% benzyl alcohol, 0-0.025% EDTA, 0-0.5% methylparaben, 0-0.5% propylparaben, 0.1-5% dimethicone, 0.5-5% PVP/eicosene copolymer, 0.1-2% Crill 6, 0.05-2% Pemulen TR-2, 0.5-5% Cremophor GS-32, 1-2% triethanolamine, 2-10% sorbitol, 0.5-10% stearic acid, and 50-90% water.

Another alternative example embodiment of the present invention is a composition for preventing moisture loss from the skin including 0-12% microspheres, 0.5-2% Germaben II, 0.0025-0.01% EDTA, 0.05-0.5% dimethicone, 3-5% Cenwax ME, 4-6.5% Perfecta, 3-5% glyceryl monostearate, 0.025-0.5% aloe gel, 1-3% triethanolamine, 2-7% glycerol, 1-3% stearic acid, 1.5-4% Paramount B, 0.15-0.75% Carbopol 2984, 2-7% urea, 0.2-0.7% fragrance, 50-75% water.

Another alternative example embodiment of the present invention is a composition for preventing moisture loss from the skin including 0-6% microspheres, 1-3% benzyl alcohol, 0.01-2% cod liver oil, 0.01-2% aloe vera lipo/aloe oil extr., 10-20% light mineral oil, 0.1-5% dimethicone, 0.95-12.5% waxes, 1-5% Arlacel 186, 15-25% sorbitol solution, 0.02-1% fragrance, 5-15% zinc oxide, and 10-68.01% water.

Another alternative example embodiment of the present invention is a composition for preventing moisture loss from the skin including 1-15% microspheres, 0.05-1% preservative, 1-7% Permethyl 101A, 0.01-2% dimethicone, 0.01-1% vitamin E, 1-10% emulsifier, 0.01-1% dl-panthenol, 1-10% glycerine, 0.1-7.5% emollient, 0.01-1% fragrance, 1-5% dihydroxyacetone, 0-0.1% citric acid, and 39.4-94.81% water.

Another alternative example embodiment of the present invention is a method of preventing moisture loss from the skin by applying to the skin a moisturizer composition, wherein the moisturizer has the composition comprising, 0-6% microspheres, 1-3% benzyl alcohol, 0.01-2% cod liver oil, 0.01-2% aloe vera lipo/aloe oil extr., 10-20% light mineral oil, 0.1-5% dimethicone, 0.95-12.5% waxes, 1-5% Arlacel 186, 15-25% sorbitol solution, 0.02-1% fragrance, 5-15% zinc oxide, and 10-68.01% water.

### DETAILED DESCRIPTION OF THE EXAMPLE EMBODIMENTS

Throughout this specification, the term “percent” is intended to mean, unless the context clearly indicates otherwise, percentages by weight. Various formulation components are identified herein by their adopted names as given by J. M. Nikitakis et. al., Eds., CTFA International Cosmetic Ingredient Dictionary, Fourth Ed., The Cosmetic, Toiletry and Fragrance Association, Washington, D.C., 1991. The functions performed by various components are listed by J. A. Wenninger et. al., Eds., CTFA Cosmetic Ingredient Handbook, Second Ed., The Cosmetic, Toiletry and Fragrance Association, Washington, D.C., 1992.

In one example embodiment, lotions and creams of the present invention may include: about 1-15% by weight of microspheres, 1-15% by weight of a long-chain fatty acid, 0.2-10% by weight of a hydrocarbon oil, 0.2-30% by weight of a silicone skin protectant, 0.5-5% by weight of an alkanolamine, 0.5-10% by weight of a humectant, 0.01-15% by weight of a preservative, 0.01-1% by weight of an antioxidant, and 50-90% by weight water. Other ingredients may be added for additional effects: for sunscreen-containing formulations, 0.5-25% by weight of a UV active can be added. For diaper creams and foot creams, however, occlusive ingredients can be decreased, but probably not completely eliminated from the formulations. The microspheres would enhance the barrier function of the occlusive ingredients so that less could be used in the formulations to achieve the same barrier function result as a composition without the microspheres, resulting in a more pleasing cream or lotion.

The long-chain fatty acid is generally a substituted or unsubstituted carboxylic acid, having about 12 to about 22 carbon atoms, and should be safe for prolonged skin contact in the formulations herein. It will be appreciated that various fatty acids may be used, e.g., lauric acid, myristic acid, palmitic acid, stearic acid, arachidic acid and tricosanoic acid.

An alkanolamine is desired for reaction with the long-chain fatty acid to form an emulsifier of the type used to produce cosmetic skin creams and lotions. Various alkanolamines may be used, e.g., ethanolamine, diethanolamine, triethanolamine, isopropanolamine, triisopropanolamine, and methylethanolamine. It will be appreciated that the long-chain fatty acid and alkanolamine contents may be adjusted to obtain the complete reaction for the emulsion.

A hydrocarbon oil or a plant or animal-derived oil may be included as skin conditioning agent to maintain an appropriate moisture level in the skin, and as a humectant to control the rate of water loss from the emulsion film as it is being applied. For example, light mineral oil, castor oil, cod liver oil, aloe oil and isodecahexane among others, or a mixture thereof, may be used. Emollients may also be included in the formulation, e.g.: oleaginous esters, ethers, and aloe extract, Hetester SSS, Cetiol OE, Lexol IPL, Performalene 400, cetyl alcohol, octyl palmitateneopentyl glycol heptanoate, neopentyl glycol diheptanoate, octyldodecyl neopentanoate, actiphyte of aloe vera, Trivent NP-13, C₁₂₋₁₅ alkyl benzoate, and mixtures thereof.

Humectants may help prevent moisture loss from the formulation as it is being applied, aiding even spreading of the lotion or cream. Humectants may be added into the formula and may include, e.g., glycerol, ethylene glycol, propylene glycol, sorbitol, mannitol, panthenol, PVM/MA decadiene crosspolymer, glyceryl polymethacrylate & propylene glycol, glyceryl polymethacrylate & propylene glycol & PVM/MA coploymer, among others, and mixtures thereof.

A silicone skin protectant may be included for its barrier properties. Barrier products may cause a greasy feel. Silicone protectants may minimize the oily feel. Oily feel may be further reduced with inclusion of an inorganic skin protectant. Silicone protectants may include, e.g., amodimethicone, cyclomethicone, hexadecyl methicone, dimethicone, vinylmethicone and the like. Inorganic skin protectants may include, e.g., calamine, kaolin, zinc oxide, titanium dioxide, zinc carbonate, iron oxide, zirconium oxide, cerium oxide and mixtures thereof.

The formulation may also include an emulsifier, e.g., silicone-based emulsifiers, glycosides, polyethylene glycols, acrylic-based emulsifiers, glyceryl monostearate, Promulgen G, Lipomulse 165, Abil WE 09, Abil EM-90, DC9011 silicone elastomer, Emulgade 68/50, Arlacel P135, Arlacel 186, Simulgel A, Simulgel EG and mixtures thereof.

The formulation may also contain preservatives. Many types are available and may be suitable for the compositions described herein, e.g., phenols, alcohols, aldehydes, dyes, surfactants, furan derivatives, quinoline, and isoquiniline derivatives, guanidines and amidines. Particularly suitable are benzyl alcohol, disodium EDTA, methylparaben, propylparaben, glyceryl dilaurate, imidasolidinyl urea, phenylethyl alcohol, benzalkonium chloride and Quarternium-15, Germaben II, Germall II, and mixtures thereof.

For a moisturizing composition which includes a sunscreen, organic UV actives can be added to the formula, e.g., octinoxate, octisdalate, homosalate, avobenzone, octocrylene, para-aminobenzoic acid, cinoxate, dioxybenzone, methyl anthralate, octocrylene, padimate O, ensulizole, sulisobenzone, trolamine salicylate, ecamsule, oxybenzone and mixtures thereof. Inorganic UV actives can also be included in the moisturizing composition, e.g., zinc oxide, titanium dioxide, iron oxide, zirconium oxide, cerium oxide, and mixtures thereof.

In some example embodiments, fragrance or odor neutralizers can be added to the formula, particularly for use in a diaper cream or foot balm. Such odor neutralizers include ORDENONE™, among others known in the art, and may be included up to about 5% by weight. A fragrance, many of which are known in the art, may also be added up to 5% by weight.

In some alternative embodiments, an insect repellant may be included in the formula. The most widely used active agent for personal care products is N,N-Diethyl-m-toluamide, frequently called “DEET” and available in the form of a concentrate containing at least about 95 percent DEET. Other synthetic chemical repellents include butyl alcohol (Stabilene), dimethyl phthalate, ethyl hexanediol, indalone, di-n-propylisocinchoronate, bicycloheptene, dicarboximide and tetrahydrofuraldehyde. Certain plant-derived materials also have insect repellent activity, including citronella oil and other sources of citronella (including lemon grass oil), limonene, rosemary oil and eucalyptus oil. Choice of an insect repellent for incorporation into the composition will frequently be influenced by the odor of the repellent. The amount of repellent agent used will depend upon the choice of agent; DEET is useful at high concentrations, such as up to about 15 percent or more, while some of the plant-derived substances are typically used in much lower amounts, such as 0.1 percent or less.

In some alternative embodiments, such as a jock-itch cream or other antifungal composition, an antifungal agent may be included in the formula. As used herein, the term “antifungal agent” refers to any compound useful as topical agents to treat fungal infections in animals (including humans). Examples of antifungal agents useful in the pharmaceutical formulations of the invention include, but are not limited to, miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, 1-ofloxacin, chlortetracycline hydrochloride, oxytetracycline hydrochloride, tetracycline hydrochloride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione and pharmaceutically acceptable salts thereof.

In example embodiments, e.g., compositions for foot creams and diaper creams or adult incontinence product, heavier oils and other waxy substances may be included, e.g., waxes, petrolatum and heavy mineral oils.

The ingredient which is critical to the improved prevention of moisture loss in the skin in the formulation of the moisturizers of the invention is a small particle called a microsphere. In particular, hollow, polyalkene/acrylic copolymeric spheres are preferred. Addition of these microspheres to any of the above suggested formulations may result in an increase in retained water on the skin, and may prevent cracking and water loss from the skin.

Using skin bioinstrumentation testing, an unexpected observation was discovered when testing a formula with an ingredient called SunSpheres™. The formula with SunSpheres™ (B) was found to significantly enhance skin barrier function compared to the same formula without SunSpheres™ (A) and compared to untreated skin (U). The effect of maintaining humidity on the skin is unique to the compositions and methods of preventing water loss from the skin described herein.

SunSpheres™ (styrene/acrylates copolymer) is an ingredient from Rohm and Haas that is provided in powder form or as a suspension. This polymer material is a hollow sphere that enables greater SPF efficiency of sunscreen products. Before this invention, there was no mention that SunSpheres™ could have an effect to enhance skin barrier function, as SunSpheres™ and other microspheres have been used primarily for their light-scattering attributes. There has been no mention that microspheres, which have been designed specifically for light-scattering purposes in the cosmetics and the paint and resin industries, could possibly be used to increase the barrier function of such cream or lotion. Some larger porous microspheres have been used for delivery of lotions, but none have been used for enhancing skin barrier function. The result described herein is surprising, and enables formulation of lotions and creams to perform a barrier function without the necessity of a layer of oily or greasy material to keep moisture in the skin, which has been the state of the art up to now. A barrier composition without or with substantially less occlusive agents is aesthetically advantageous, as well as useful for retaining and maintaining skin moisture, especially in such areas as for foot balms and diaper creams, where the integrity of the skin is at risk for irritation and cracking.

SunSpheres™ are inert hollow spheres of a very small particle size (about 325 nm). The material is thought to enhance SPF by increasing light scattering efficiency in the presence of a sunscreen and is advertised, promoted, and used for that purpose. According to Rohm and Haas, a “rough calculation demonstrated that because of the particle size and density of the SunSpheres™ product, there are about 10 to 20 trillion particles (scattering centers) per weight percent of solid polymer product added to a formulation. Having this large a number of particles in a sunscreen film or other cosmetic product (which concentrate 4 to 5 times as the film dries) allows for efficient scattering of UV radiation through the film, thereby increasing the path-length.” Although SunSpheres™ can concentrate on the skin's surface as part of a sunscreen film, the inventors believe that it was not previously known that the SunSpheres™ would have an effect on enhancing skin barrier function for at least three factors. In particular, SunSpheres™ are advertised as having been “proven to have minimal to no effect on water resistant formulations” (see data from Rohm and Haas web site, http://www.rhpersonalcare.com, accessed Nov. 14, 2007). It was previously believed that SunSpheres™ do not influence how a formula interacts with the skin surface. Moreover, data collected from a cosmetic efficacy study showed that the formula with SunSpheres™ (B) was not significantly better than the formula without SunSpheres™ (A) at hydrating skin (see Corneometer data, Table 2).

### EXAMPLES

In the examples, “Sunspheres™ powder” refers to styrene/acrylates copolymer added as a solid. “Sunspheres™ suspension” refers to styrene/acrylates copolymer added as part of a liquid, containing 25.0-26.0% solids in water solvent. Thus, Sunspheres™ suspension added at approximately 11% by weight is equivalent to adding Sunspheres™ powder at approximately 3% by weight.

### Example 1

Two sunscreen-containing compositions were compared to test the water retention in the skin after application of the formulae, and compared with untreated skin. The compositions tested were as listed in Table 1, below.

TABLE 1
Sunscreen
Formulations

| Category | Description | A | B |
|---|---|---|---|
| antioxidant | dl-alpha tocopherol | 0.05000 | 0.05000 |
| preservative | Benzyl alcohol | 0.50000 | 0.50000 |
| | disodium EDTA | 0.01000 | 0.01000 |
| | methylparaben, NF | 0.20000 | 0.20000 |
| | propylparaben, NF | 0.10000 | 0.10000 |
| UV active | avobenzone | 3.00000 | 3.00000 |
| | Octisalate, USP (Sunarome) | 5.00000 | 5.00000 |
| | Octocrylene, USP | 2.00000 | 2.00000 |
| | Homosalate, USP (Coppertone) | 13.00000 | 13.00000 |
| | oxybenzone, USP | 4.0000 | 4.0000 |
| silicone | Dow corning 200 fluid, 350 CST | 0.50000 | 0.50000 |
| emulsion | PVP/Eicosene copolymer | 2.00000 | 2.00000 |
| stabilizer | | | |
| emulsifier | Crill 6 | 0.71000 | 0.71000 |
| hydrocarbon | Cremophor GS-32 (castor oil) | 0.29000 | 0.29000 |
| oil | | | |
| emulsifier | Pemulen TR-2 | 0.10000 | 0.10000 |
| alkanolamine | triethanolamine, 99% NF | 0.90000 | 0.90000 |
| humectant | sorbitol solution, 70% USP | 5.00000 | 5.00000 |
| long-chain | stearic acid, NF triple pressed | 1.20000 | 1.20000 |
| fatty acid | | | |
| | Sunspheres ™ powder | | 3.00000 |
| | USP purified water | 61.54000 | 58.54000 |

This single-blinded, controlled kinetic study was conducted by an independent laboratory to assess the cosmetic efficacy of two products to deliver moisture to the skin after a single application by use of clinical grading (visual and tactile), a Corneometer, and TEWL measurements. The Corneometer quantifies the moisture content of the stratum corneum using an electrical capacitance method. An increase in measured capacitance indicates an increase in skin surface hydration. Trans-epidermal water loss (TEWL) is measured using a DermaLab meter in conjunction with a computer to determine skin surface integrity. For this measurement, a decrease in TEWL indicates an improvement in barrier function.

Twenty-five female subjects completed the study. Subjects did not shave their legs for at least two days prior to the study start and refrained from applying any moisturizing topical products to the lower legs for at least five days prior to the study start. Subjects qualified for study participation by having lower leg dry skin scores of 2-4 on a scale of 0-4 and lower leg tactile roughness scores of 3-8 on a scale of 0-9. A total of three test sites were marked on the lateral side of each subject's lower legs (two sites on one leg and one site on the other leg). The test products and untreated control were assigned to test sites according to a pre-determined randomization scheme. The test products were shaken prior to each dose to ensure proper mixing. Each test site was clinically graded for dryness and tactile roughness and triplicate Corneometer measurements were taken prior to test material application (baseline), immediately post-application (approximately 15 minutes), and 1, 4, and 8 (±10 minutes) hours post-application. TEWL measurements were taken at each test site prior to test site application (baseline), immediately post-application (approximately 15 minutes), and 1, 4, and 8 (±10 minutes) hours post-application. Test materials (A) and (B) were very effective in improving skin dryness, tactile roughness and skin hydration as measured by Corneometry when compared to mean baseline measures. An analysis of variance (ANOVA) comparison showed that test materials A and B were at parity for skin hydration at the immediate, 1 hour, and 8 hour time points, while test material A significantly outperformed test material B for skin hydration at the 4 hour time point. (See Corneometry data, Table 2.)

TABLE 2
Corneometer Results

| Formula | Baseline | Immediate | 1 Hour | 4 Hour | 8 Hour |
|---|---|---|---|---|---|
| A | 24.3 | 40.7* | 39.5* | 39.0*+ | 35.8* |
| B | 25.2 | 40.5* | 39.5* | 36.6* | 34.3* |
| U | 24.4 | 25.9* | 26.7* | 24.6 | 25.2 |

A = formula without SunSpheres ™
B = formula with SunSpheres ™
U = untreated skin
*= statistically significant (p ≦ 0.05) improvement compared to baseline
+= values are statistically significant (p ≦ 0.05) compared to B and U

However, ANOVA comparisons showed that test material B significantly outperformed test material A for TEWL at all time points measured, indicating that test material B enhanced stratum corneum barrier function (see TEWL data, Table 3).

TABLE 3
TEWL Measurements

| Formula | Baseline | 1 Hour | 4 Hour | 8 Hour |
|---|---|---|---|---|
| A | 4.8 | 3.2* | 3.1* | 3.2* |
| B | 5.24 | 2.9*+ | 2.9*+ | 2.97* |
| U | 5.16 | 3.8* | 3.6* | 3.8* |

A = formula without SunSpheres ™
B = formula with SunSpheres ™
U = untreated skin
*= statistically significant (p ≦ 0.05) improvement compared to baseline
+= values are statistically significant compared to A and U

Based on results from this study, formula B with SunSpheres™ is believed to have an ability to enhance stratum corneum barrier function. This benefit can be directly attributed to the SunSpheres™, because an identical formula without SunSpheres™ (A) was significantly less effective in enhancing stratum corneum barrier function.

### Example 2

A foot cream can be prepared using a formulation including microspheres but with the addition of less occlusive agent than without, thereby making it more pleasant to use, and enable application to be smoother. Two formulations are given in Table 4, one with, and one without SunSpheres™, with the expectation that TEWL measurements could demonstrate the difference in moisture retention in a test similar to that in Example 1.

TABLE 4
Foot Cream

| | | % weight | % weight |
|---|---|---|---|
| | | with | without |
| Category | | micro- | micro- |
| Formula | Description | spheres | spheres |
| microspheres | SunSpheres ™ suspension | 11.10 | |
| preservative | Germaben II | 1.00 | 1.00 |
| preservative | EDTA | 0.05 | 0.05 |
| silicone | dimethicone | 0.10 | 0.10 |
| wax | Cenwax ME | 4.00 | 4.00 |
| petrolatum | Perfecta | 5.35 | 5.35 |
| emulsifier | Glyceryl monostearate | 4.00 | 4.0 |
| emollient | aloe gel | 0.05 | 0.05 |
| alkanolamine | triethanolamine, 99% NF | 1.40 | 1.40 |
| humectant | glycerol | 5.00 | 5.00 |
| long-chain | stearic acid, NF triple pressed | 2.00 | 2.00 |
| fatty acid | | | |
| | Paramount B | 2.50 | 2.50 |
| rheology | Carbopol 2984 | 0.35 | 0.35 |
| modifier | | | |
| skin | urea | 5.00 | 5.00 |
| softener | | | |
| fragrance | Peach SZ-9600 | 0.40 | 0.40 |
| water | USP purified water | 57.70 | 68.80 |

A foot cream of this composition would be expected to have the following characteristics:

1) smooth application due to the presence of the microspheres

2) enhanced barrier function of the humectant, wax and petrolatum due to the microspheres

3) less oily feel because there are less occlusive ingredients necessary due to the presence of the microspheres

4) enhanced skin barrier function, allowing less moisture to pass through the skin to the environment.

The higher percent of microspheres in this formulation would be expected to enhance the ability of the composition to maintain or prevent water loss.

### Example 3

A diaper cream or adult incontinence product can be formulated using SunSpheres™ to enhance the barrier function properties of the waxes and oils normally included. If desired, decreased amounts of these ingredients can be used resulting in a less greasy cream that is easier to apply. Two formulations are given in Table 5, one with, and one without, SunSpheres™, with the expectation that TEWL measurements could demonstrate the difference in moisture retention in a test similar to that in Example 1.

TABLE 5
Diaper Cream/Adult Incontinence Product

| | | % weight | % weight |
|---|---|---|---|
| | | with | without |
| Category | | micro- | micro- |
| Formula | Description | spheres | spheres |
| microspheres | SunSpheres ™ powder | 3.00 | |
| preservative | benzyl alcohol | 2.00 | 2.00 |
| hydrocarbon oil | cod liver oil | 0.05 | 0.05 |
| | aloe vera lipo/aloe oil extr. | 0.05 | 0.05 |
| | Light mineral oil | 15.00 | 15.00 |
| silicone | Dow Corning 360 Medical | 1.00 | 1.00 |
| | Fluid | | |
| wax | Ozokerite Wax SP-1021P | 2.00 | 2.00 |
| | paraffin wax | 1.00 | 1.00 |
| | synth. Beeswax | 1.50 | 1.50 |
| emulsifier | Arlacel 186 | 3.00 | 3.00 |
| humectant | sorbitol solution | 20.00 | 20.00 |
| fragrance | frag baby powder TCS23179 | 0.20 | 0.20 |
| antimicrobial/ | zinc oxide | 10.00 | 10.00 |
| antifungal | | | |
| water | purified water | 42.98 | 45.98 |

A diaper cream or adult incontinence product of this composition would be expected to have the following characteristics:
1) smooth application due to the presence of the microspheres
2) enhanced barrier function of the humectant, wax and petrolatum due to the microspheres
3) less oily feel because there are less occlusive ingredients necessary due to the presence of the microspheres
4) enhanced skin barrier function, allowing less moisture to pass through the skin to the environment.

### Example 4

A sunless tanning lotion can also be formulated using SunSpheres™. It can be envisioned that the microspheres will aid in the application of such a lotion, as well as maintaining moisture within the stratum corneum while the skin coloring agent acts. This is important for achieving a smooth, non-dry sunless tan. Two formulations containing SunSpheres™ are listed in Table 6 below.

TABLE 6
Sunless Tanning Lotion

| Category | | % weight | % weight |
|---|---|---|---|
| Formula | Description | A | B |
| microspheres | SunSpheres ™ | 3.00 | 12.00 |
| preservative | Germall II | 0.20 | 0.20 |
| | EDTA | 0.05 | 0.05 |
| hydrocarbon oil | Permethyl 101A | 5.00 | 5.00 |
| silicone | Dow Corning 200 Fluid | 1.00 | 1.00 |
| antioxidant | vitamin E | 0.50 | 0.50 |
| emulsifier | Promulgen G | 3.00 | 3.00 |
| | Lipomulse 165 | 3.00 | 3.00 |
| humectant | dl-panthenol | 0.10 | 0.10 |
| | glycerin | 3.00 | 3.00 |
| emollient | Hestester SSS | 2.00 | 2.00 |
| | cetyl alcohol | 1.00 | 1.00 |
| | Performalene 400 | 0.25 | 0.25 |
| fragrance | #73422B | 0.35 | 0.35 |
| coloring agent | dihydroxyacetone | 3.00 | 3.00 |
| | citric acid | 0.01 | 0.01 |
| water | purified water | 75.04 | 66.04 |

In formulation A, addition of SunSpheres™ powder is exemplified. In formulation B, SunSpheres™ suspension is exemplified.

A sunless tanning lotion of this composition would be expected to have the following characteristics:

1) smooth application due to the presence of the microspheres

2) enhanced barrier function of the humectant and hydrocarbon oil due to the microspheres

3) less oily feel because there are less occlusive ingredients necessary due to the presence of the microspheres

4) enhanced skin barrier function, allowing less moisture to pass through the skin to the environment.

### Methods of Use

The compositions described in the example embodiments of the present invention may be used for the prevention and treatment of skin dryness, cracking, flaking, etc. The compositions may be applied to the skin as a lotion, cream, spray, or as a component of another skin care product such as a cosmetic. The application may be as needed, e.g., every four hours, twice daily, or daily. In the example diaper cream or adult incontinence product, after the skin area is cleaned, and the cream is applied to the public or perianal area, the cream and the skin may be covered by a diaper, child's training pants, rubber pants, or a conventional undergarment. In the case of the example foot balm or foot cream, after the cream is applied the foot may be covered with a sock, shoe, or other footwear article.

### Alternative Embodiments

While some of the example embodiments discussed above discuss a particular type of microsphere, it will be recognized this particular type of microsphere is merely exemplary, and that a range of other microsphere types and sizes may be employed.

In the preceding specification, the present invention has been described with reference to specific example embodiments thereof. It will, however, be evident that various modifications and changes may be made thereunto without departing from the broader spirit and scope of the present invention as set forth in the claims that follow. The specification and drawings are accordingly to be regarded in an illustrative rather than restrictive sense.

What is claimed is:

1. A composition for preventing moisture loss from the skin, comprising:
a moisturizing component; and
microspheres combined with the moisturizing component, wherein the composition does not have UV organic actives.

2. A diaper cream according to claim 1, further comprising: zinc oxide up to 25% by weight.

3. A sunless tanning lotion according to claim 1, further comprising: a skin coloring agent.

4. A sunless tanning lotion of claim 3, wherein the skin coloring agent is dihydroxyacetone.

5. A foot balm according to claim 1, further comprising urea.

6. A jock-itch cream according to claim 1, further comprising an antifungal agent.

7. The composition of claim 1, wherein said microspheres comprise copolymers of polyalkenes and acrylates.

8. The composition of claim 7 wherein said microspheres further comprise copolymers of styrene and methacrylate.

9. The composition for preventing moisture loss as in claim 8 wherein said microspheres are hollow.

10. The composition for preventing moisture loss as in claim 9 wherein said microspheres have an average particle size of 325 nm.

11. The composition for preventing moisture loss as in claim 1 wherein the moisturizing composition reduces loss of water from the skin as measured by TEWL as compared to an identical composition without the microspheres.

12. The composition for preventing moisture loss as in claim 11 wherein the moisturizing composition comprising microspheres results in sustained lower TEWL measurements over time than a moisturizing composition without microspheres.

13. The composition for preventing moisture loss as in claim 1, wherein said microspheres are present at a concentration range of about 1% to about 15% by weight.

14. The composition for preventing moisture loss as in claim 13, wherein said microspheres are present at a concentration range of about 2% to about 3% by weight.

15. The composition for preventing moisture loss as in claim 1 wherein said moisturizing component further comprises:
a long-chain fatty acid, a hydrocarbon oil, a silicone, a humectant, a preservative, an antioxidant, an emulsifier, an emulsion stabilizer, an alkanolamine, and water.

16. The composition for preventing moisture loss as in claim 15 wherein the long-chain fatty acid is selected from the group consisting of lauric acid, myristic acid, palmitic acid, stearic acid, arachdic acid and tricosanoic acid.

17. A method of preventing moisture loss as in claim 15 wherein the hydrocarbon oil is selected from the group consisting of light mineral oils, castor oil, cod liver oil, aloe oil, isodecahexane and Cremophor GS-32, and mixtures thereof.

18. The composition for preventing moisture loss as in claim 15 wherein the silicone is selected from the group consisting of amodimethicone, cyclomethicone, hexadecyl methicone, dimethicone, and vinyl methicone, Dow Corning 200 fluid 350CST, and mixtures thereof.

19. The composition for preventing moisture loss as in claim 15 wherein the humectant is selected from the group consisting of glycerol, ethylene glycol, propylene glycol, sorbitol, mannitol, PVM/MA decdiene crosspolymer, glyceryl polymethacrylate & propylene glycol, and glyceryl polymethacrylate & propylene glycol & PVM/MA copolymer, and mixtures thereof.

20. The composition for preventing moisture loss as in claim 15 wherein the preservative is selected from the group consisting of phenols, alcohols, aldehydes, dyes, surfactants, furan derivatives, quinoloine, isoquinoline derivatives, quanidines, amidines, benzyl alcohol, disodium EDTA, methylparaben, propylparaben, glyceryl dilaurate, imidasolidinyl urea, phenylethyl alcohol, benzalkonium chloride and Quarternium-15 and mixtures thereof.

21. The composition for preventing moisture loss as in claim 15 wherein the antioxidant said moisturizing composition is selected from the group consisting of vitamin E, dl-alpha tocopherol, vitamin E acetate, and ascorbyl palmitate.

22. The composition for preventing moisture loss as in claim 15 wherein the emulsifier is selected from the group consisting of silicone-based emulsifiers, glycosides, polyethylene glycols, acrylic-based emulsifiers, Crill 6, Pemulen TR-2, Abil WE 09, Abil EM-90, DC9011 silicone elastomer, Emulgade 68/50, Arlacel P135, Simulgel A, Simulgel EG and mixtures thereof.

23. The composition for preventing moisture loss as in claim 15 wherein the emulsion stabilizer of said moisturizing composition is selected from the group consisting of PVP/eicosene copolymer and mixtures thereof.

24. The composition for preventing moisture loss as in claim 15 wherein the alkanolamine of said moisturizing composition is selected from the group consisting of ethanolamine, diethanolamine, triethanolamine, isopropanolamine, triisopropanolamine, methylethanolamine, and mixtures thereof.

25. The composition for preventing moisture loss as in claim 15 wherein the emollient of said moisturizing composition is selected from the group consisting of oleaginous esters, ethers, and aloe extract, Hetester SSS, Cetiol OE, Lexol IPL, Performalene 400, cetyl alcohol, octyl palmitateneopentyl glycol heptanoate, neopentyl glycol diheptanoate, octyldodecyl neopentanoate, actiphyte of aloe vera, Trivent NP-13, C₁₂₋₁₅ alkyl benzoate, and mixtures thereof.

26. The composition for preventing moisture loss as in claim 1, wherein said moisturizing composition further comprises an insect repellant.

27. The composition of claim 26, wherein the insect repellant selected from the group consisting of DEET, butyl alcohol, dimethyl phthalate, ethyl hexanediol, indalone, di-n-propylisocinchoronate, bicycloheptene, dicarboximide and tetrahydrofuraldehyde.

28. The composition for preventing moisture loss as in claim 1, further comprising 0-1% odor neutralizer or fragrance.

29. A composition for preventing moisture loss from the skin, comprising: 0-3% microspheres, 0.01-1% dl-alpha tocopherol, 0.5-5% benzyl alcohol, 0-0.025% EDTA, 0-0.5% methylparaben, 0-0.5% propylparaben, 0.1-5% dimethicone, 0.5-5% PVP/eicosene copolymer, 0.1-2% Crill 6, 0.05-2% Pemulen TR-2, 0.5-5% Cremophor GS-32, 1-2% triethanolamine, 2-10% sorbitol, 0.5-10% stearic acid, and 50-90% water.

30. A composition for preventing moisture loss from the skin, comprising: 0-12% microspheres, 0.5-2% Germaben II, 0.0025-0.01% EDTA, 0.05-0.5% dimethicone, 3-5% Cenwax ME, 4-6.5% Perfecta, 3-5% glyceryl monostearate, 0.025-0.5% aloe gel, 1-3% triethanolamine, 2-7% glycerol, 1-3% stearic acid, 1.5-4% Paramount B, 0.15-0.75% Carbopol 2984, 2-7% urea, 0.2-0.7% fragrance, 50-75% water.

31. A composition for preventing moisture loss from the skin, comprising: 0-6% microspheres, 1-3% benzyl alcohol, 0.01-2% cod liver oil, 0.01-2% aloe vera lipo/aloe oil extr., 10-20% light mineral oil, 0.1-5% dimethicone, 0.95-12.5% waxes, 1-5% Arlacel 186, 15-25% sorbitol solution, 0.02-1% fragrance, 5-15% zinc oxide, and 10-68.01% water.

32. A composition for preventing moisture loss from the skin, comprising: 1-15% microspheres, 0.05-1% preservative, 1-7% Permethyl 101A, 0.01-2% dimethicone, 0.01-1% vitamin E, 1-10% emulsifier, 0.01-1% dl-panthenol, 1-10% glycerine, 0.1-7.5% emollient, 0.01-1% fragrance, 1-5% dihydroxyacetone, 0-0.1% citric acid, and 39.4-94.81% water.

33. A method of preventing moisture loss from the skin by applying to the skin a moisturizer compositions according to claim 1, wherein the moisturizer has the composition comprising, 0-6% microspheres, 1-3% benzyl alcohol, 0.01-2% cod liver oil, 0.01-2% aloe vera lipo/aloe oil extr., 10-20% light mineral oil, 0.1-5% dimethicone, 0.95-12.5% waxes, 1-5% Arlacel 186, 15-25% sorbitol solution, 0.02-1% fragrance, 5-15% zinc oxide, and 10-68.01% water.

34. A method of preventing moisture loss from the skin by applying to the perianal area of the skin a moisturizer according to claim 1.

35. The method of claim 34, further comprising:
covering the perianal area of the skin with a diaper.

36. A method of preventing moisture loss from the skin by applying to the skin a moisturizer composition according to claim 1 and covering the area of skin with an article of clothing.

37. The method of claim 36, wherein the article of clothing is an article of footwear.
